(19) United States
(12) Patent Application Publication
(10) Pub. No.: US 2007/0054259 A1
(43) Pub. Date: Mar. 8, 2007

(54) Modulation of hnRNP H and treatment of DM1

(75) Inventors: Dongho Kim, Los Angeles, CA (US); John J. Rossi, Alta Loma, CA (US)

Correspondence Address: ROTHWELL, FIGG, ERNST & MANBECK, P.C., 1425 K STREET, N.W., SUITE 800, WASHINGTON, DC, 20005, US

(73) Assignee: City of Hope, Duarte, CA (US)

(21) Appl. No.: 11/453,260

(22) Filed: Jun. 15, 2006

Related U.S. Application Data
(60) Provisional application No. 60/691,232, filed on Jun. 17, 2005.

(51) Int. Cl. C12Q 1/00; C12Q 1/68

(52) U.S. Cl. 435/4; 435/6

(57) ABSTRACT

The present invention is directed to the discovery that the heterogeneous nuclear ribonucleprotein H (hnRNP H) is capable of binding mutant myotonic dystrophy (DM) protein kinase (DMPK) mRNA. The present invention is also directed to the discovery that modulation of the expression of hnRNP H results in reduced nuclear retention of the mutant DMPK mRNA. The present invention is further directed to screening compounds to identify drugs useful for treating DM type 1 (DM1).

### CROSS-REFERENCE TO RELATED APPLICATION

The present application is related to and claims priority under 35 U.S.C. § 119(e) to U.S. provisional patent application Ser. No. 60/691,232 filed 17 Jun. 2005, incorporated herein by reference.

This application was made with Government support under Grant Nos. AU29329 and HL074704 funded by the National Institutes of Health, Bethesda, Md. The federal government may have certain rights in this invention.

### BACKGROUND OF THE INVENTION

The present invention is directed to the discovery that the heterogeneous nuclear ribonucleprotein H (hnRNP H) is capable of binding mutant myotonic dystrophy (DM) protein kinase (DMPK) mRNA. The present invention is also directed to the discovery that modulation of the expression of hnRNP H results in reduced nuclear retention of the mutant DMPK mRNA. The present invention is further directed to screening compounds to identify drugs useful for treating DM type 1 (DM1).

The publications and other materials used herein to illuminate the background of the invention, and in particular, cases to provide additional details respecting the practice, are incorporated by reference, and for convenience are referenced in the following text by author and date and are listed alphabetically by author in the appended bibliography.

Myotonic dystrophy type 1 (DM1) is an autosomal, dominantly inherited neuromuscular disorder with a global incidence of 1 per 8000 (Harper, 2001). Adult onset DM1 is primarily characterized by myotonia, muscle wasting, and weakness, but also affects a number of organs and results in cataracts, cardiac conduction abnormalities, testicular atrophy, male baldness, and insulin resistance (Harper, 2001). The mutation responsible for the disease is a (CUG)n repeat expansion in the 3′ un-translated region of the DM protein kinase (DMPK) gene (Mahadevan et al., 1992; Fu et al., 1992; Brook et al., 1992). This repeat ranges in size from 5-37 repeats in the normal population to between 50-1000 repeats in adult onset cases (Harper, 2001).

Among several proposed molecular mechanisms, the RNA dominant mutational model proposes that triplet repeat expansion causes a gain-of-function at the RNA level (Tapscott, 2000; Filippova et al., 2001), possibly by sequestering essential cellular RNA binding proteins (Caskey et al., 1996; Timchenko and Caskey, et al., 1996; Miller et al., 2000; Fardaei et al., 2002). Targeting and destruction of mutant DMPK mRNA releases these factors thus allowing restoration of several of the normal myotube functions (Furling et al., 2003; Langlois et al., 2003). In support of the gain of function model, transgenic mice containing CUG repeats in an unrelated mRNA display myotonia and a myopathy phenotype (Mankodi et al., 2000). Mice transgenic for the human DMPK region with expanded CTG repeats display muscular and brain abnormalities (Seznec et al., 2001). Several features of DM1 pathogenesis can be explained by aberrant alternative-splicing defects (Faustino and Cooper, 2003). Misregulation of insulin receptor (IR) (Savkur et al., 2001), muscle-specific chloride channel (CLC-1) (Charlet et al., 2002; Mankodi et al., 2002) and cardiac troponine T (cTNT) (Philips et al., 1998) splicing is linked with common symptoms of DM1 such as insulin resistance, skeletal muscle membrane hyperexcitability characteristic of myotonia and cardiac conduction defects (Savkur et al., 2001; Furling et al., 1999).

Several CUG repeat binding proteins have been identified to date (Miller et al., 2000; Tian et al., 2000; Lu et al., 1999; Timchenko et al., 1996; Timchenko et al., 1999; Bhagwati et al., 1996; Kino et al., 2004). CUG-BP1 is one of the first CUG binding proteins identified. While this protein does not co-localized with the nuclear foci formed by mutant DMPK transcripts, it has been shown that expression levels of CUG-BP1 are increased in DM1 (Timchenko et al., 1996; Roberts et al., 1997). Functional analyses indicate that increased expression of CUG-BP1 could be implicated for the aberrant regulation of cTNT, IR, and CIC-1 by binding to U/G rich motifs in introns adjacent to the regulated splice site (Timchenko et al., 2004; Timchenko et al., 2001a; Timchenko et al., 2001b). Muscleblind (MBNL) protein family members in humans have also been shown to bind to CUG repeats and can also co-localize with the nuclear foci (Fardaei et al., 2002; Ho et al., 2004; Dansithong et al., 2005; Kanadia et al., 2003; Fardaei et al., 2001). Recently, a muscleblind (MBNL1) knock-out mouse was produced that displayed muscle, eye, and RNA splicing abnormalities that are characteristic of DM1 disease (Kanadia et al., 2003). Although MBNL1 protein depletion in mice helps explain some of the molecular mechanism involved in DM1, it is reasonable to hypothesize that there are additional CUG binding factors which work coordinately with these aforementioned CUG binding proteins.

To address this possibility, we utilized a modified RNA/protein crosslinking assay to search for proteins that bind DM1 derived CUG repeat containing transcripts. This assay identified the heterogeneous nuclear ribonucleprotein H (hnRNP H) as a novel protein capable of binding RNA with CUG repeats when a branch point sequence is located downstream. HnRNP H is best known for its role as an alternative splicing factor and in pre-mRNA cleavage and polyadenylation. (Buratti, et al., 2004; Caputi, et al. 2002; Chen et al. 1999; Arhin, et al., 2002; Bagga, 1998) Surprisingly, we show that knock-down of endogenous hnRNP H expression by SiRNAs in cells expressing an EGFP gene fused to CUG repeats leads to release of nuclear sequestrated transcripts and restoration of EGFP expression. These results could provide insight into the mechanisms implicated in the nuclear sequestration of mutant DMPK transcripts in DM1.

Mutant DMPK mRNAs containing the trinucleotide expansion are retained in the nucleus of DM1 cells and form discrete foci. The nuclear sequestration of RNA binding proteins and associated factors binding to the CUG expansions is believed to responsible for several of the splicing defects observed in DM1 patients and could ultimately be linked to DM1 muscular pathogenesis. Several RNA binding proteins capable of co-localizing with the nuclear-retained mutant DMPK mRNAs have already been identified but none can account for the nuclear retention of the mutant transcripts.

Thus, it is desired to identify and isolate RNA binding proteins that bind to mutant DMPK-derived RNA. The identification of such proteins a factor capable of binding and possibly modulating nuclear retention of mutant DMPK mRNA is an important link in the understanding of the molecular mechanisms that lead to DM1 pathogenesis. The identification of such proteins also provides additional targets for the development of drugs for treating DM1.

### SUMMARY OF THE INVENTION

The present invention is directed to the discovery that the heterogeneous nuclear ribonucleprotein H (hnRNP H) is capable of binding mutant DMPK mRNA. The specific binding of hnRNP H was found to require not only a CUG repeat expansion but also a splicing branch point distal to the repeats. The present invention is further directed to the discovery that modulation of the expression of hnRNP H results in reduced nuclear retention of the mutant DMPK mRNA. This latter discovery was demonstrated by rescued protein expression from RNA with CUG repeat expansions resulting from the suppression of hnRNP H expression by RNAi. The present invention is further directed to screening compounds to identify drugs useful for treating DM1.

Thus, in a first aspect, the present invention provides the identification of a protein that binds mutant DMPK mRNA and sequesters the mutant DMPK in the nucleus.

In a second aspect, the present invention provides the discovery that modulation of the expression of hnRNP H results in reduced nuclear retention of the mutant DMPK mRNA.

In a third aspect, the present invention provides methods for screening candidate compounds to identify drugs useful in treating DM1.

### BRIEF DESCRIPTION OF THE FIGURES

FIGS. 1A-1C show UV cross linking of CUG repeat RNAs in HeLa nuclear extracts. FIG. 1A: RNA clones used. The fragments of the DMPK gene with 5 (SEQ ID NO:1), 46 (SEQ ID NO:2), or 85 CTG (SEQ ID NO:3) repeats were cloned and transcribed in vitro with T7 RNA polymerase. Black bars represent the vector sequence common to all three clones. The 3′ branch site is underlined. (CUG)85′ (SEQ ID NO:4) is the clone with 85 repeats of CUG repeats and a mutated branch site. FIG. 1B: UV crosslinking using HeLa nuclear extracts. Lane 1, CUG5; lane 2, CUG85; lane 3 CUG 46; lane 4, biotinylated CUG46 (underlined); lane 5, with biotinylated CUG85 (underlined). FIG. 1C: UV crosslinking in DM extracts. Lanes 1 and 2, HeLa total cell extracts; lanes 3 and 4, total DM1 cell extracts before cell differentiation; lanes 5 and 6, DM1 extracts after differentiation.

FIGS. 2A-2D show the purification and identification of the CUG repeat binding protein. FIG. 2A: Purification of the binding protein. The eluted proteins from the RNA affinity column using the CUG46 or CUG85 RNAs were separated in a SDS-P AGE gel. FIG. 2B: UV crosslinking assays in extracts treated with pre-immune (pre) or hnRNP H anti-sera (post). FIG. 2C: The crosslinking products were treated with pre-immune sera (pre, lane 2) or anti-hnRNP H (post, lane 3). FIG. 2D: The levels of hnRNP Hand beta-actin were compared from cells that were mock transfected or transfected with anti-hnRNPH siRNAs (Top panel). The siRNA treated cell extracts were used in UV crosslinking assays (Bottom panel).

FIGS. 3A-3C show that an additional cellular factor(s) are required for dimer formation of hnRNP H. FIG. 3A: Recombinant hnRNP H does not dimerize by itself. A UV crosslinking assay was carried out using CUG46 or CUG85 RNAs incubated in total He La cell extracts (lanes 1 and 2) or with recombinant hnRNP H (lanes 3 and 4). FIG. 3B: A cellular factor is required for hnRNP H dimerization. Lane 1, CUG85 RNA alone; lane 2, CUG85 RNA incubated with total HeLa cell extract; lane 3, CUG85 RNA incubated with a hnRNP H-depleted HeLa cell extract; lane 4, CUG85 RNA incubated with 10 ng of recombinant hnRNP H; lane 5, CUG85 RNA with hnRNP H immuno-depleted extract to which 10 ng of purified recombinant hnRNP H was added prior to CUG85 RNA addition. To confirm immuno-depletion of hnRNP H, the total amount of hnRNP H was compared prior to (lane 2, bottom panel) and following (lane 3, bottom panel) immunodepletion. FIG. 3C: Recombinant MBNL1 has no effect on hnRNP H-mediated complex formation. Lane 1, CUG85 RNA alone; lane 2, CUG85 RNA incubated with total cell extract; lane 3, CUG85 RNA incubated with 100 ng of recombinant hnRNP H; lane 4, CUG85 RNA incubated with 100 ng of recombinant MBNL1; lane 5, CUG85 RNA incubated with 500 ng of MBNL1; lane 6, CUG 85 RNA incubated with 10 ng of hnRNP Hand 500 ng of MBNL1.

FIGS. 4A and 4B show that the binding of hnRNP H to CUG repeats is proportional to the length of the repeats and requires the 3′ splicing branch site of ex on 16. FIG. 4A: lane 1, CUG5 RNA only, lane 2, CUG5 RNA with total cell extract, lane3, CUG5 with recombinant hnRNP H, lane 4, CUG46 RNA only, lane 5, CUG46 and total extract, lane 6, CUG46 and recombinant hnRNP H, lane 7, CUG85 RNA only, lane 8; CUG85 and total extract, lane 9, CUG85 and recombinant hnRNP H. FIG. 4B: Binding requires the 3′ branch site. Lane 1, CUG85 clone alone; lane 2, CUG85 incubated in the total cell extract, lane 3, the RNA was incubated in the presence of 10 ng of recombinant hnRNP H, lane 4, CUG85 RNA with the mutated 3′ branch site, lane 5, the mutant RNA with 10 ng of recombinant hnRNP H protein.

FIGS. 5A and 5B show that RNA foci of DM1 cells contain hnRNP H. FIG. 5A: Co-localization assay for hnRNP Hand RNA foci in DM1 cell. First column, immuno-staining of endogenous hnRNP H; second column, in situ hybridization with a CAG 10 probe; third column, superimposed images using a double filter. FIG. 5B: HnRNP H interacts with CUG repeats in vivo. DM1 myoblast extracts were crosslinked by UV irradiation. HnRNP H in the total extract was immuno-purified using the hnRNP H antibody. HnRNP H-associated RNAs were extracted and resolved in a denaturing gel, blotted to a nylon membrane and probed with a 32P labeled CAG1O DNA (Top panel). Lane 1, the extract prepared from the UV irradiated cells was treated with pre-immune sera; lane 2, extract from non-irradiated cells was treated with anti-hnRNP H antibody; lane 3, extract from irradiated cells treated with anti-hnRNP H antisera. To monitor the immuno-purification procedure, an aliquot of the treated samples was analyzed by Western blotting (Bottom panel).

FIGS. 6A-6C show that the suppression of hnRNP H expression can rescue the nuclear retention of RNA with CUG repeats. FIG. 6A: HEK 293T cells were transfected with either the eGFP-(CUG)5 (Panel 1) or the EGFP-(CUG)85 (Panel 2) reporter genes alone. An irrelevant siRNA (Panel 3) or an anti-hnRNP H siRNA (Panel 4) were co-transfected with the eGFP-(CUG)85 reporter. FIG. 6B: SiRNA-mediated gene-specific knock-down of hnRNP H. (See panels of FIG. 6A for lane identities). FIG. 6C: SiRNA mediated expression knockdown of hnRNP can restore expression of the eGFP-(CUG)85 reporter gene in primary myoblasts. The left panel shows transfection of myoblasts with an irrelevant siRNA, the right panel shows expression of the reporter in myoblasts transfected with the anti-hnRNP H siRNA.

FIGS. 7A and 7B show that hnRNP F is not required for the binding of hnRNP H and CUG repeats. FIG. 7A: Northern analyses for the level of hnRNP F after treatment of the scrambled siRNA (C) or hnRNP F siRNA (F). The unidentified band (*) was used as an internal control. FIG. 7B: total cell extract was made from each siRNA treated cells and used in the crosslinking assay.

### DETAILED DESCRIPTION OF THE INVENTION

The present invention is directed to the discovery that the heterogeneous nuclear ribonucleprotein H (hnRNP H) is capable of binding mutant DMPK mRNA. The specific binding of hnRNP H was found to require not only a CUG repeat expansion but also a splicing branch point distal to the repeats. The present invention is also directed to the discovery that modulation of the expression of hnRNP H results in reduced nuclear retention of the mutant DMPK mRNA. This latter discovery was demonstrated by rescued protein expression from RNA with CUG repeat expansions resulting from the suppression of hnRNP H expression by RNAi. The present invention is further directed to screening compounds to identify drugs useful for treating DM1.

In one embodiment, the present invention is directed to the identification of a protein that binds mutant DMPK mRNA and sequesters the mutant DMPK in the nucleus. In accordance with the present invention, a modified RNA/protein crosslinking assay was used to search for proteins that bind DM1 derived CUG repeat containing transcripts. As described in further detail in the Examples, this assay identified the heterogeneous nuclear ribonucleprotein H (hnRNP H) as a novel protein capable of binding RNA with CUG repeats when a branch point sequence is located downstream. HnRNP H is best known for its role as an alternative splicing factor and in pre-mRNA cleavage and polyadenylation (Buratti et al., 2004; Caputi and Zahler, 2002; Chen et al., 1999; Arhin et al., 2002; Bagga et al., 1998).

In a second embodiment, the present invention is directed to the discovery that modulation of the expression of hnRNP H results in reduced nuclear retention of the mutant DMPK mRNA. It was surprisingly found, as shown in the Examples, that knock-down of endogenous hnRNP H expression by siRNAs in cells expressing an enhanced green fluorescent protein (eGFP) gene fused to CUG repeats leads to release of nuclear sequestrated transcripts and restoration of eGFP expression.

The discovery of hnRNP H as a protein that binds mutant DMPK and the discovery that modulation of the expression of hnRNP H leads to release of nuclear sequestrated transcripts and restoration of expression provide insight into the mechanisms implicated in the nuclear sequestration of mutant DMPK transcripts in DM1 and provides a drug target for discovering drugs useful for treating DM1. Thus, in a third aspect, the present invention provides methods for screening candidate compounds to identify drugs useful in treating DM1. Thus, in certain embodiments, the invention provides methods for identifying agents which modulate the activity of hnRNP H, and preferably agents that modulate the interaction (whether direct or indirect) between hnRNP H and mutant DMPK. Accordingly, the invention provides screening methods for identifying therapeutics. A therapeutic of the invention can be any type of compound, including a protein, a peptide, a proteoglycan, a polysaccharide, a peptidomimetic, a small molecule, and a nucleic acid. A nucleic acid can be, e.g., a gene, an antisense nucleic acid, a ribozyme, an interfering RNA (such as an siRNA), or a triplex molecule. Therapeutics can be identified using various assays depending on the type of compound and activity of the compound that is desired. Set forth below are at least some assays that can be used for identifying therapeutics. It is within the skill of the art to design additional assays for identifying therapeutics.

In vitro systems can also be used to identifying compounds that inhibit, activate or bind to proteins encoded by a gene of interest. The identified compounds may be useful, for example, in modulating the activity of wild type and/or mutant gene products. In vitro systems may also be utilized to screen for compounds that disrupt normal regulatory interactions.

Cell based assays can be used, in particular, to identify compounds which modulate expression of the hnRNP H gene, modulate translation of the mRNA encoding hnRNP H, modulate the posttranslational modification of the core protein of hnRNP H, modulate the stability of the mRNA or protein or modulate the binding of hnRNP H with mutant DMPK. Accordingly, in one embodiment, a cell which is capable of producing hnRNP H, e.g., a differentiated myoblast, is incubated with a test compound and the amount of hnRNP H produced in the cell medium is measured and compared to that produced from a cell which has not been contacted with the test compound. The specificity of the compound vis-a-vis hnRNP H can be confirmed by various control analysis, e.g., measuring the expression of one or more control genes. In a second embodiment, a cell which is capable of producing hnRNP H is transfected with a gene encoding a reporter or marker protein in which the gene contains GUC repeat extensions. The transfected cell is incubated with a test compound. The restoration of expression of the reporter or marker protein in the treated transfected cell is compared to a transfected cell which has not been contacted with the test compound. In a third embodiment, the effect of a test compound on transcription of the hnRNP H gene is determined by transfection experiments using a reporter or marker gene operatively linked to at least a portion of the promoter of the hnRNP H gene. A promoter region of a gene can be isolated, e.g., from a genomic library according to methods known in the art.

The reporter or marker gene can be any gene encoding a protein which is readily quantifiable. Such proteins include enzymes, such as β-galactosidase, luciferase, chloramphenicol acytransferase, β-glucuronidase and alkaline phosphatase, that can produce specific detectable products, and proteins that can be directly detected, such as green fluorescent protein or enhanced green fluorescent protein (eGFP). Many additional reporter proteins are known and have been used for similar purposes. In addition, virtually any protein can be directly detected by using, for example, specific antibodies to the protein. eGFP (Zhang et al., 1996) is a preferred reporter or marker protein. Additional markers (and associated antibiotics) that are suitable for either positive or negative selection of eukaryotic cells are disclosed, inter alia, in Sambrook and Russell (2001), Molecular Cloning, 3^{rd} Ed., Cold Spring Harbor Laboratory Press, Cold Spring Harbor, N.Y., and Ausubel et al. (1992), Current Protocols in Molecular Biology, John Wiley & Sons, including periodic updates. Any of the disclosed markers, as well as others known in the art, may be used to practice the present invention.

Assays used to identify compounds that bind to proteins involve preparing a reaction mixture of a given protein and the test compound under conditions and for a time sufficient to allow the two components to interact and bind, thus forming a complex which can be removed and/or detected in the reaction mixture. The protein used can vary depending upon the goal of the screening assay. For example, where agonists of the natural ligand are sought, a full length protein, or a fusion protein containing a protein or polypeptide that affords advantages in the assay system (e.g., labeling, isolation of the resulting complex, etc.) can be utilized. In addition, in vitro assays may involve substances, enzymes, ant the like which are secreted from myoblasts, which are then assayed.

The screening assays can be conducted in a variety of ways. For example, one method to conduct such an assay would involve anchoring the protein, polypeptide, peptide or fusion protein or the test substance onto a solid phase and detecting binding between the protein and test compound or mutant cell. In one embodiment of such a method, the receptor protein reactant may be anchored onto a solid surface, and the test compound, which is not anchored, may be labeled, either directly or indirectly. In another embodiment of the method, the test protein is anchored on the solid phase and is complexed with labeled antibody (and where a monoclonal antibody is used, it is preferably specific for a given region of the protein). Then, a test compound could be assayed for its ability to disrupt the association of the protein/antibody complex.

Alternatively, a reaction can be conducted in a liquid phase, the reaction products separated from unreacted components, and complexes detected; e.g., using an immobilized antibody specific for the test protein, polypeptide, peptide or fusion protein, or the test compound to anchor any complexes formed in solution, and a labeled antibody specific for the other component of the possible complex to detect anchored complexes.

The basic principle of the assay systems used to identify compounds that interfere with the interaction between a protein and its binding partner or partners involves preparing a reaction mixture containing the test protein, polypeptide, peptide or fusion protein as described above, and the binding partner under conditions and for a time sufficient to allow the two to interact and bind, thus forming a complex. In order to test a compound for inhibitory activity, the reaction mixture is prepared in the presence and absence of the test compound. The test compound can be initially included in the reaction mixture, or may be added at a time subsequent to the addition of the test protein and its binding partner. Control reaction mixtures are incubated without the test compound or with a placebo. The formation of any complexes between the test protein and the binding partner is then detected. The formation of a complex in the control reaction, but not in the reaction mixture containing the test compound, indicates that the compound interferes with the interaction of the test protein and the binding partner.

Further details concerning the above described in vitro systems and additional in vitro systems can be found in U.S. Pat. No. 6,080,576.

A variety of test compounds can be evaluated in accordance with the present invention. In certain embodiments, the compounds to be tested can be derived from libraries (i.e., are members of a library of compounds). While the use of libraries of peptides is well established in the art, new techniques have been developed which have allowed the production of mixtures of other compounds, such as benzodiazepines (Bunin and Ellman, 1992; DeWitt et al., 1993), peptoids (Zuckermann, 1994), oligocarbamates (Cho et al., 1993), and hydantoins (DeWitt et al., 1993). An approach for the synthesis of molecular libraries of small organic molecules with a diversity of 104-105 as been described (Carell et al., 1994a; Carell et al., 1994b).

The compounds of the present invention can be obtained using any of the numerous approaches in combinatorial library methods known in the art, including: biological libraries; spatially addressable parallel solid phase or solution phase libraries, synthetic library methods requiring deconvolution, the ‘one-bead one-compound’ library method, and synthetic library methods using affinity chromatography selection. The biological library approach is limited to peptide libraries, while the other four approaches are applicable to peptide, non-peptide oligomer or small molecule libraries of compounds (Lam, 1997). Other exemplary methods for the synthesis of molecular libraries can be found in the art, for example in Erb et al. (1994), Horwell et al. (1996) and Gallop et al. (1994).

Libraries of compounds may be presented in solution (e.g., Houghten et al., 1992), or on beads (Lam et al., 1991), chips (Fodor et al., 1993), bacteria (U.S. Pat. No. 5,223,409), spores (U.S. Pat. No. 5,223,409), plasmids (Cull et al., 1992) or on phage (Scott and Smith, 1990; Devlin et al., 1990; Cwirla et al., 1990; Felici et al., 1991). In still another embodiment, the combinatorial polypeptides are produced from a cDNA library.

Exemplary compounds which can be screened for activity include, but are not limited to, peptides, nucleic acids, carbohydrates, small organic molecules, and natural product extract libraries.

The goal of rational drug design is to produce structural analogs of biologically active polypeptides of interest or of small molecules with which they interact (e.g., agonists, antagonists, inhibitors) in order to fashion drugs which are, for example, more active or stable forms of the polypeptide, or which, e.g., enhance or interfere with the function of a polypeptide in vivo. Several approaches for use in rational drug design include analysis of three-dimensional structure, alanine scans, molecular modeling and use of anti-id antibodies. These techniques are well known to those skilled in the art. Such techniques may include providing atomic coordinates defining a three-dimensional structure of a protein complex formed by said first polypeptide and said second polypeptide, and designing or selecting compounds capable of interfering with the interaction between a first polypeptide and a second polypeptide based on said atomic coordinates.

Following identification of a substance which modulates or affects polypeptide activity, the substance may be further investigated. Furthermore, it may be manufactured and/or used in preparation, i.e., manufacture or formulation, or a composition such as a medicament, pharmaceutical composition or drug. These may be administered to individuals.

A substance identified as a modulator of polypeptide function may be peptide or non-peptide in nature. Non-peptide “small molecules” are often preferred for many in vivo pharmaceutical uses. Accordingly, a mimetic or mimic of the substance (particularly if a peptide) may be designed for pharmaceutical use.

The designing of mimetics to a known pharmaceutically active compound is a known approach to the development of pharmaceuticals based on a “lead” compound. This approach might be desirable where the active compound is difficult or expensive to synthesize or where it is unsuitable for a particular method of administration, e.g., pure peptides are unsuitable active agents for oral compositions as they tend to be quickly degraded by proteases in the alimentary canal. Mimetic design, synthesis and testing is generally used to avoid randomly screening large numbers of molecules for a target property.

Once the pharmacophore has been found, its structure is modeled according to its physical properties, e.g., stereochemistry, bonding, size and/or charge, using data from a range of sources, e.g., spectroscopic techniques, x-ray diffraction data and NMR. Computational analysis, similarity mapping (which models the charge and/or volume of a pharmacophore, rather than the bonding between atoms) and other techniques can be used in this modeling process. Such techniques include those disclosed in U.S. Pat. No. 6,080,576.

A template molecule is then selected, onto which chemical groups that mimic the pharmacophore can be grafted. The template molecule and the chemical groups grafted thereon can be conveniently selected so that the mimetic is easy to synthesize, is likely to be pharmacologically acceptable, and does not degrade in vivo, while retaining the biological activity of the lead compound. Alternatively, where the mimetic is peptide-based, further stability can be achieved by cyclizing the peptide, increasing its rigidity. The mimetic or mimetics found by this approach can then be screened to see whether they have the target property, or to what extent it is exhibited. Further optimization or modification can then be carried out to arrive at one or more final mimetics for in vivo or clinical testing.

With regard to intervention, any compounds which reverse any aspect of a given phenotype or expression of any gene in vivo and which modulates protein activity or binding with binding partner in vitro should be considered as candidates for further development or potential use in humans. Dosages of test agents may be determined by deriving dose-response curves using methods well known in the art.

This invention further pertains to agents identified by the above-described screening assays and uses thereof for treating DM1. Pharmaceutical compositions containing an identified agent as the active ingredient can be prepared according to conventional pharmaceutical compounding techniques. See, for example, Remington: The Science and Practice of Pharmacy, 21st Ed., Lippincott Williams & Wilkins, Philadelphia, 2005. Typically, a therapeutically effective amount of an active ingredient is admixed with a pharmaceutically acceptable carrier. By a “therapeutically effective amount” or simply “effective amount” of an active compound is meant a sufficient amount of the compound to treat the desired condition at a reasonable benefit/risk ratio applicable to any medical treatment. The actual amount administered, and the rate and time-course of administration, will depend on the nature and severity of the condition being treated. Prescription of treatment, e.g. decisions on dosage, timing, etc., is within the responsibility of general practitioners or specialists, and typically takes account of the disorder to be treated, the condition of the individual patient, the site of delivery, the method of administration and other factors known to practitioners. Examples of techniques and protocols can be found in Remington: The Science and Practice of Pharmacy.

The carrier may take a wide variety of forms depending on the form of preparation desired for administration, e.g., intravenous, oral, parenteral, intramuscular, subcutaneous or intrathecal. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. For examples of delivery methods see U.S. Pat. No. 5,844,077, incorporated herein by reference.

Wetting agents, emulsifiers and lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. Examples of pharmaceutically acceptable antioxidants include, but are not limited to, water soluble antioxidants such as ascorbic acid, cysteine hydrochloride, sodium bisulfite, sodium metabisulfite, sodium sulfite, and the like; oil soluble antioxidants, such as ascorbyl palmitate, butylated hydroxyanisole (BHA), butylated hydroxytoluene (BHT), lecithin, propyl gallate, aloha-tocopherol and the like; and the metal chelating agents such as citric acid, ethylenediamine tetraacetic acid (EDTA), sorbitol, tartaric acid, phosphoric acid and the like.

Exemplary methods for administering compounds (e.g., so as to achieve sterile or aseptic conditions) will be apparent to the skilled artisan. Certain methods suitable for administering compounds useful according to the present invention are set forth in Goodman and Gilman's The Pharmacological Basis of Therapeutics, 11th Ed. (2006). The administration to the patient can be intermittent; or at a gradual, continuous, constant or controlled rate. Administration can be to a warm-blooded animal (e.g. a mammal, such as a mouse, rat, cat, rabbit, dog, pig, cow or monkey); but advantageously is administered to a human being. Administration occurs after general anesthesia is administered. The frequency of administration normally is determined by an anesthesiologist, and typically varies from patient to patient.

The pharmaceutical compositions will generally contain from about 0.0001 to 99 wt. %, preferably about 0.001 to 50 wt. %, more preferably about 0.01 to 10 wt. % of the active ingredient by weight of the total composition. In addition to the active agent, the pharmaceutical compositions and medicaments can also contain other pharmaceutically active compounds. Examples of other pharmaceutically active compounds include, but are not limited to, analgesic agents, cytokines and therapeutic agents in all of the major areas of clinical medicine. When used with other pharmaceutically active compounds, the agents may be delivered in the form of drug cocktails. A cocktail is a mixture of any one of the compounds useful with this invention with another drug or agent. In this embodiment, a common administration vehicle (e.g., pill, tablet, implant, pump, injectable solution, etc.) would contain both the instant composition in combination supplementary potentiating agent. The individual drugs of the cocktail are each administered in therapeutically effective amounts. A therapeutically effective amount will be determined by the parameters described above; but, in any event, is that amount which establishes a level of the drugs in the area of body where the drugs are required for a period of time which is effective in attaining the desired effects.

The practice of the present invention employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., Molecular Cloning (Cold Spring Harbor Laboratory Press, Cold Spring Harbor, N.Y., 1982); Sambrook et al., Molecular Cloning, 2nd Ed. (Cold Spring Harbor Laboratory Press, Cold Spring Harbor, N.Y., 1989); Sambrook and Russell, Molecular Cloning, 3rd Ed. (Cold Spring Harbor Laboratory Press, Cold Spring Harbor, N.Y., 2001); Ausubel et al., Current Protocols in Molecular Biology (John Wiley & Sons, updated through 2005); Glover, DNA Cloning (IRL Press, Oxford, 1985); Anand, Techniques for the Analysis of Complex Genomes, (Academic Press, New York, 1992); Guthrie and Fink, Guide to Yeast Genetics and Molecular Biology (Academic Press, New York, 1991); Harlow and Lane, Antibodies, (Cold Spring Harbor Laboratory Press, Cold Spring Harbor, N.Y., 1998); Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, (Blackwell Scientific Publications, Oxford, 1988); Hogan et al., Manipulating the Mouse Embryo, (Cold Spring Harbor Laboratory Press, Cold Spring Harbor, N.Y., 1986); Westerfield, M., The zebrafish book. A guide for the laboratory use of zebrafish (Danio rerio), 4the Ed., (Univ. of Oregon Press, Eugene, Oreg., 2000).

### EXAMPLES

The present invention is described by reference to the following Examples, which are offered by way of illustration and are not intended to limit the invention in any manner. Standard techniques well known in the art or the techniques specifically described below were utilized.

### Example 1

### Materials and Methods

DNA Clones

The DMPK clone containing CUG 100 repeats (pRMK-100) was digested with SacI and EcoRI restriction endonucleases and cloned into the pGEM vector and further modified by deletion of a SacI/SacII fragment. Among several subclones containing a variety of CUG repeats, the clones containing 46 and 85 CUG repeats were selected following sequence confirmation. (CUG)85′ which is the (CUG)85 clone with mutated 3′ branch site was created by PCR using two primers (5′ GAACGGGGCTCGAAGCTTCCTT 3′ (SEQ ID NO:5) and 5′ CTAGACTGGAATTCGGCTTATGGTCACTGATC 3′ (SEQ ID NO:6) and cloned into the pBluescript II SK vector. RNAs were transcribed in vitro using T7 RNA polymerase on linerized DNA plasmid templates in a 20 μl reaction. For the generation of biotinylated RNA, 10% of the UTP in the transcription reaction was replaced with biotinylated UTP (Roche) in a 100 ul reaction. RNA produced from the transcription reaction was mixed with 4 volumes of sterile water and further purified using a MicroSpin™-G50 column (Amersham Biosciences, Piscataway, N.J.). To create the hnRNP H-EGFP fusion gene, hnRNP H was amplified by PCTR using two primers (5′ CAGCCATATGCTCGAGTGATG 3′ (SEQ ID NO:7) and 5′ CTTTGT TAGCAGCCGGATCC 3′ (SEQ ID NO:8)) and the produced cloned into the XhoI/BamHI sites of the pLEGFP-CI vector (Clonetech).

Extract Preparation and hnRNH H Purification

The total cell extracts of HeLa and DM1 cells were prepared as following. Cells (1×10⁸) were harvested and washed with buffer D sequentially mixed 100 ul of buffer D, and sonicated for 15 sec at 4° C. After 5 min of micro-centrifugation at 4° C., the supernatants were collected and used as total cell extracts. The HeLa nuclear extract was used for the purification of CUG binding proteins. The extract was precipitated with 30% Ammonium sulfate. The supernatants were harvested following a 10 min centrifugation at 4° C. 300 ul of extract were incubated with 300 ul of pre-washed Streptavidin M-280 Dynabeads for 30 minutes at room temperature. The extracts were recovered by microcentrifugation and used for purification of CUG binding proteins. The pre-treated extract was incubated with 30 μg of E. coli tRNA for 20 minutes at room temperature, and 30 μg of biotinylated CUG RNAs were mixed and incubated for additional 30 minutes. The buffer D pre-washed beads were incubated for 30 minutes and washed again with 1 ml of buffer D 3 times. The bound proteins were eluted by buffer D containing 200 mM KCl. The eluted proteins were separated in 15% SDS-PAGE gel. The 50 kDa fragment was gel purified and sequenced by mass spectrometry in the protein microsequencing facility of the City of Hope. Recombinant hnRNP H protein was purified as described by others (Markovtsov et al., 2000).

Immunodepletion of hnRNP H

100 μl of anti-hnRNP H antiserum was incubated with 200 μl of Protein A conjugated Dynabeads at room temperature for 1 hour. The beads were washed with 1 ml of PBS 5 times and incubated with 50 μl of the nuclear extract at 4° C. for 1 hour. The mixture was spun for 5 min, and the supernatant was used as the hnRNP H immuno-depleted extract. The depletion of hnRNP H was confirmed by Western blotting with an anti-hnRNP H antisera. For the reconstituted extract, the 20 μl of the hnRNP H depleted extract was mixed with 20 ng of recombinant hnRNP H protein and incubated at room temperature for 10 min.

In Vitro and In Vivo Cross Linking Assay

A total of 10 μl of extract was mixed with E. coli tRNA (Sigma) at a final concentration of 2 mg/ml and incubated at room temperature for 10 min. 1 μl of G50 column purified labeled RNA was mixed and incubated at room temperature for 20 min. The reaction mixture was pipetted into a petri dish maintained at 4° C. on ice cold water. UV crosslinking was performed using a UV Stratalinker 2400 (Stratagene, San Diego, Calif.) 5 cm from the light source for 10 min. The irradiated samples were digested with 10 μg of RNAse A for 10 min at 37° C. and resolved in a 10 or 15% SDS-PAGE gel. For UV crosslinking in the presence of the anti hnRNP H antibody, 2 μl of the antisera were mixed with the extract for 10 minutes prior to the crosslinking treatment. For in vivo UV cross linking, DM1 cells were placed on petri plates and irradiated by UV as described above. Extracts were prepared following sonication and incubated in the presence of antibody conjugated Protein A conjugated Dynabeads as described in the section for the immunodepletion assay.

Native Gel Assays

Nondenaturing composite gel electrophorsis was performed as described (Kim et al., 1999). The reaction was mixed with an equal volume of non-denaturing loading dye containing bromophenol blue and 50% glycerol.

RNAi Assay

For synthesis of the siRNA targeting hnRNP H, two sets of oligos were designed (AS1: 5′ AAGGTGGAGAGGGATTCGTGGCCTGTCTC 3′ (SEQ ID NO:9), S1: 5′ AACCA CGAATCCCTCTCCACCCCTGTCTC 3′ (SEQ ID NO:10). siRNA was synthesized using the Silencer siRNA construction kit from Ambion (Austin, Tex.). For siRNA assays, 293T or DM1 cells that were 30% confluent were transfected with 100 ng of the EGFP-(CUG)85 reporter gene and the anti-hnRNP H siRNA or a scrambled siRNAs (IDT, Coralville, Iowa) in a final concentration of 10 nM using Lipofectamine 2000 (Invitrogen, Carlsbad, Calif.). The suppression of EGFP expression was tested 24 hours later. For suppression of hnRNP H, the cells were harvested after 72 hours followed by total RNA isolation. For knock down of hnRNP F, a Dicer substrate dsRNA (Kim, D. H., 2005) (sense 5′GUUAGGAACAUUUUGAG UUACUUGAA 3′ (SEQ ID NO:11), and antisense; 5′UUCAAGUAACUCAAAUGUUCCUA ACAA 3′ (SEQ ID NO:12)) was and transfected into HEK293T cells at a final concentration of 20 nM described above. Three days later cells were harvested and divided into two aliquots. One aliquot was used to prepare total RNA for Northern blot assays and the other for preparation of a total cell extract. For Northern blot analyses, a total of a 20 μg of RNA were loaded in each well of a 1% agarose gel and the RNAs were electorphoresed and blotted onto a nylon membrane (Hybond). For hybridization of hnRNP F specific oligonucleotide (5′ AAGTAACTCAAATGTTCCTAACAA 3′ (SEQ ID NO:13)) was used.

Primary Human Muscle Cell Cultures

DM1, CDM1 and normal control myoblasts were obtained from the quadriceps of 15 week-old aborted fetuses. The DM1 fetus had approximately 750 CUG repeats (verified by Southern blot analysis). Skeletal muscle biopsies were approved by Laval University and the CHUL's ethical committees. Myoblasts were grown in MCDB-120 supplemented with 15% heat-inactivated fetal bovine serum, 5 μg/ml insulin, 0.5 mg/ml BSA, 10 ng/ml human hrEGF, 0.39 μg/ml dexamethasone, 50 μg/ml streptomycin and 50 μg/ml penicillin. Differentiation was carried out in DMEM supplemented with 10 μg/ml insulin, 10 μg/ml apo-transferrin, 50 μg/ml streptomycin and 50 μg/ml penicillin.

In Situ Hybridization

Detection of foci was performed using 10 ng of a Cy3 labeled (CAG)10 oligo (IDT, Coralville, Iowa) as described (Teneja, K. L. 1998). Detection by immunoflurorescence involved using two antibodies (1^{st}; and anti-hnRNP H, 2^{nd}; anti-rabbit antibody conjugated with FITC) as described previously (O'Brien et al., 1994).

### Example 2

### UV CrossLinking of a Mutant DMPK 3′ UTR Binding Protein

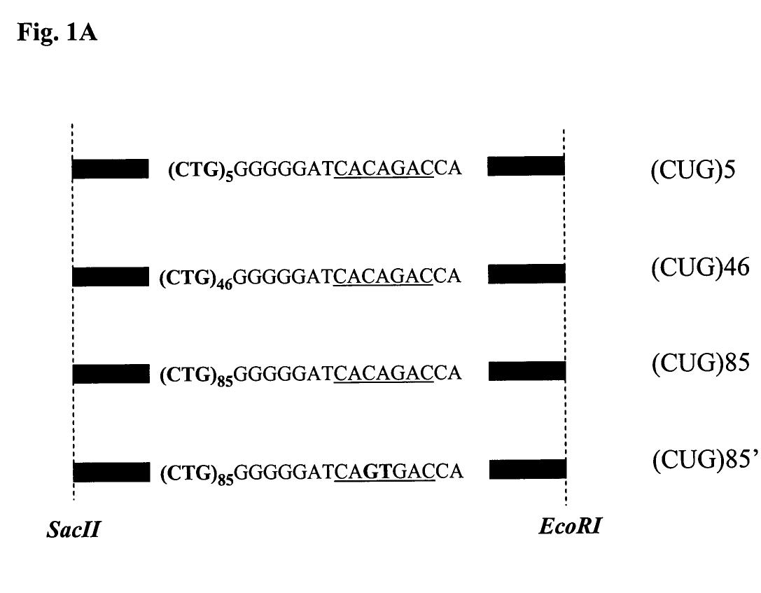

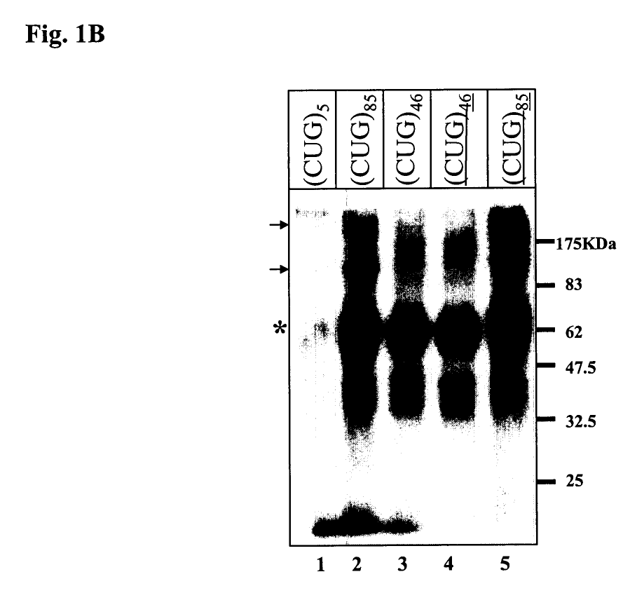

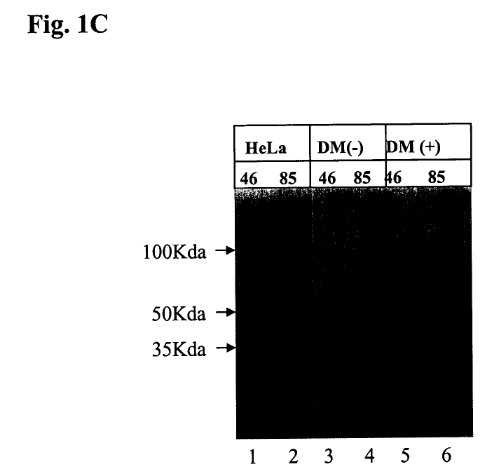

The probes used in the in vitro crosslinking assays consisted of a partial sequence of the region of the DMPK gene and contained either (CUG)5, (CUG)46, or (CUG)85 DMPK repeats and a 3′ splicing branch site (FIG. 1A). The DNAs were linearized and used as templates for in vitro transcription. The radioactively labeled RNA was incubated in a HeLa cell extract followed by UV crosslinking. We were unable to detect specific crosslinking products when the DMPK RNA with a 5 CUG repeats ((CUG)5) was used for the assays, despite a previous report that several proteins bind to the CUG elements (FIG. 1B. Lane1) (Tiscornia and Mahadevan, 2000). In contrast to (CUG)5 RNA, when the (CUG)46 RNA was used as a probe, we obtained a crosslink to a 50 kDa protein indicated by * (lane 3). Interestingly, the amount of crosslinking to this protein was increased using the CUG85 RNA probe (lane 2). Additional products were also observed, with approximate molecular weights of 100 and 200 kDa, (indicated by the arrowhead in FIG. 1B). To aid in the purification of the crosslinked protein(s), we tested crosslinking to RNAs containing biotinlylated UTP. The binding patterns of unmodified and biotinylated RNAs were identical, demonstrating that the biotinylated RNA could be used for purification of the bound protein(s) (lanes 4 and 5).

To further insure of the potential biological relevance of the 50 kDa protein in DM1, we performed the UV crosslinking assay using extracts prepared from fetal DM1 myoblasts containing 750 repeats (DM1) (FIG. 1C). Extracts were adjusted to similar concentrations prior to the crosslinking assays. The 50 kDa cross-linked products were observed with the CUG85 probe but not with the CUG46 when the DM1 cell extract was used (FIG. 1C, lanes 3 and 4). We did see however observe binding of a 35 kDa protein to both HeLa and DM1 extracts using the CUG46 probe. Because the 35 kDa protein did not bind to the longer CUG repeats, it was not investigated further.

### Example 3

### HnRNP H Can Bind and Dimerize in Presence of Expanded CUG Repeats

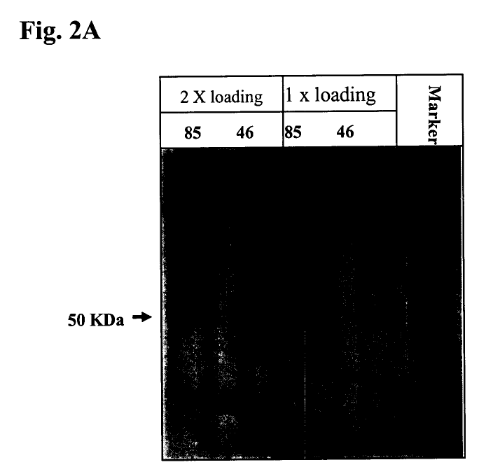

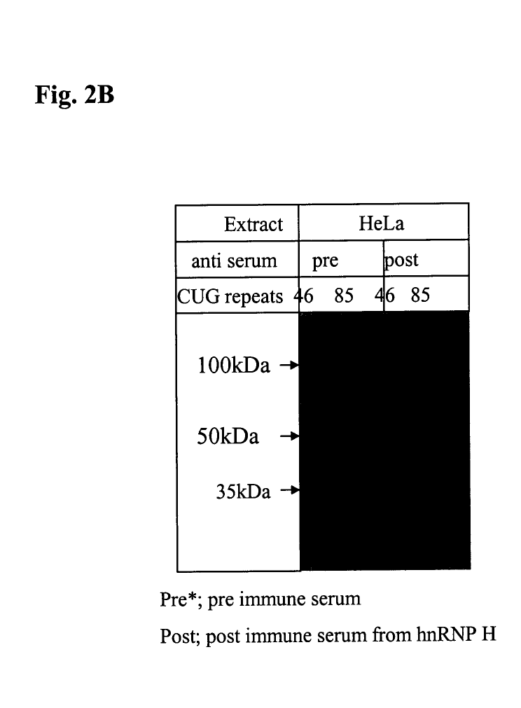

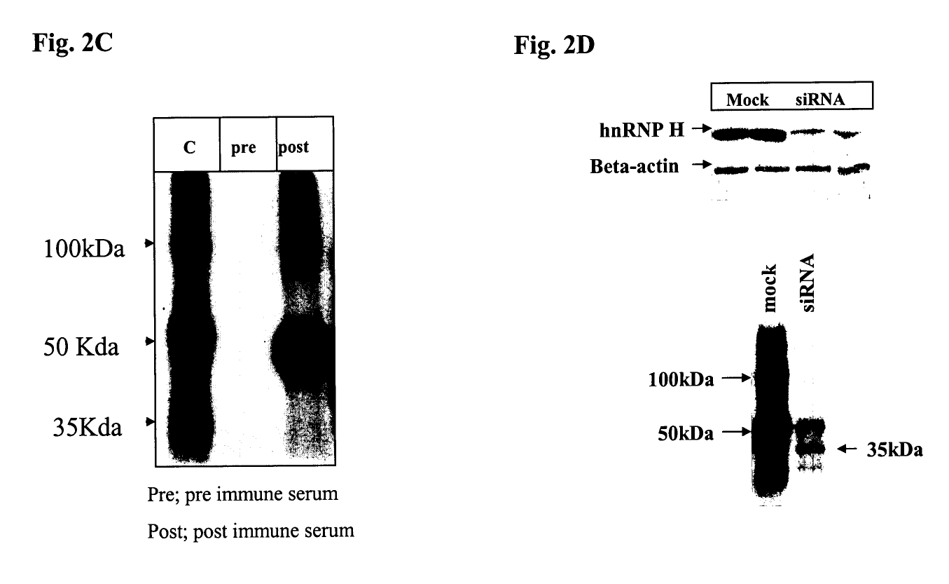

To identify the 50 kDa protein, we prepared a nuclear HeLa cell extract, and the interacting protein was further purified from the extract using affinity purification with biotinylated (CUG)85 RNA. The RNA/protein complex was purified using strepavidin conjugated beads and the bound proteins were washed and eluted in high salt. The 50 kDa protein eluted in 200 mM salt (FIG. 2A). The relative differences in eluted 50 kDa protein from the CUG46 versus CUG85 RNAs may reflect differences in the binding efficiencies to these two different substrates. The proteins eluted from the biotinylated CUG substrates were excised from the SDS-PAGE gel, eluted and micro-sequenced. The sequences we obtained from three peptides were each derived from hnRNP H (Table 1, for 50 kDa).

TABLE 1

Peptides Identified by Mass Spectrometry

| | Identified hnRNP H Peptide |
|---|---|
| Sequenced Protein | Sequences (SEQ ID NO:) |
| 50 kDa | STGEAFVQFASQEIAEK (14) |
| | HTGPNSPDTANDGFVR (15) |
| | YGDGGSTFQSTTGHCVHMR (16) |
| | VHIEIGPGR (17) |
| | DLNYCFSGMSDHR (18) |
| | VHIEIGPDGR (19) |
| 100 kDa | YVEVFK (20) |
| | DLNYCFSGMSDHR (21) |
| | VHIEIGPDGR (22) |

Anti-hnRNP H antibodies (a generous gift from Drs. Black and Helfman) were used to confirm the identify of the CUG binding protein (FIG. 2B). HeLa cell extracts were treated with either pre-immune and post-immune antisera UV crosslinking assays performed on the post immune sera treated extracts demonstrated loss of the 50 kDa and 100 kDa bands and revealed enhanced binding of the 35 kDA protein (FIG. 2B). Seemingly, there is competitive binding of hnRNP H and the 35 kDA protein for the (CUG)85 RNA (and to a lesser extent to the (CUG)46) RNA). The specific inhibition of the band at 100 kDa may is suggestive of dinner formation by hnRNP H on this template.

To investigate this hypothesis, we performed immuno-precipitations using hnRNP H antiserum-conjugated beads in HeLa cell extracts UV-crosslinked to the (CUG)85 probe (FIG. 2C). Bands migrating at 50 kDa and 100 kDa were generated from this crosslinking whereas the 33 kDA protein was absent, demonstrating the specificity of the antibody. The larger 100 kDa product was then purified and micro-sequenced. As observed previously with the 50 kDa band, all of the sequenced peptides are derived from hnRNP H (Table 1, lower row). To eliminate the possibility of cross-contamination of the 100 kDa protein with the abundant 50 kDa product, the crosslinking was repeated using HeLa cell extracts prepared from cells treated with an anti-hnRNP H siRNA (FIG. 2D). If the 100 kDa product is indeed a dimer of hnRNP H, then its expression should also be knocked-down. Northern gel analyses of hnRNP H mRNA in cells treated with the anti-hnRNP H siRNA versus a mock siRNA (scrambled) showed a significant reduction in the amount of hnRNP H mRNA (FIG. 2D, top panel). Extracts were prepared from the siRNA-transfected cells and tested in the crosslinking assay (FIG. 2D, lower panel). Both the 50 kDa and 100 kDa products were strongly reduced demonstrating that the 100 kDa crosslinking product requires hnRNP H formed when the (CUG)85 RNA is used as bait. Interestingly, binding of the unidentified 35 kDa protein was also restored when hnRNP H was depleted from the extracts.

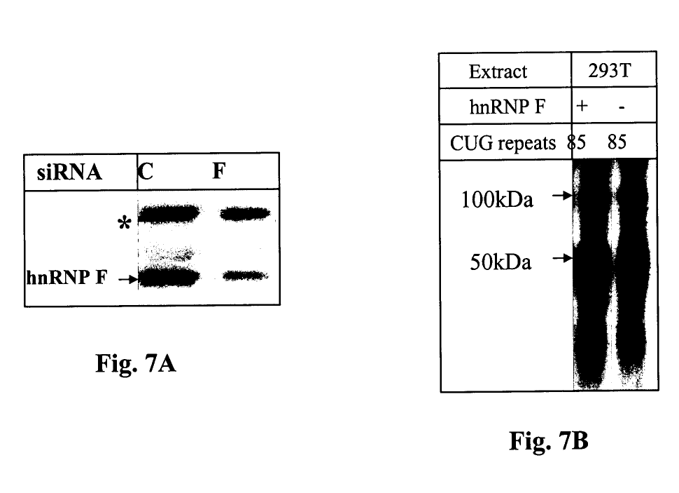

It has been shown that hnRNP H and F interact to form a heterodimer (Chou et al. 1999). Although our data suggest that hnRNP H itself may dimeize on the longer repeat template (Table 1, FIGS. 2C and D), it is still possible that the 100 kDA complex is comprised of the two proteins. To test whether or not this is the case, RNAi was used to reduce the level hnRNP F. Although the RNA level ofhnRNP F was reduced by 80% (FIG. 7A), we did not observe an effect on the relative amount of the 100 kDa product in the UV crosslinking assay (FIG. 7B).

### Example 4

### HnRNP H Dimerization Requires Additional Cellular Factors

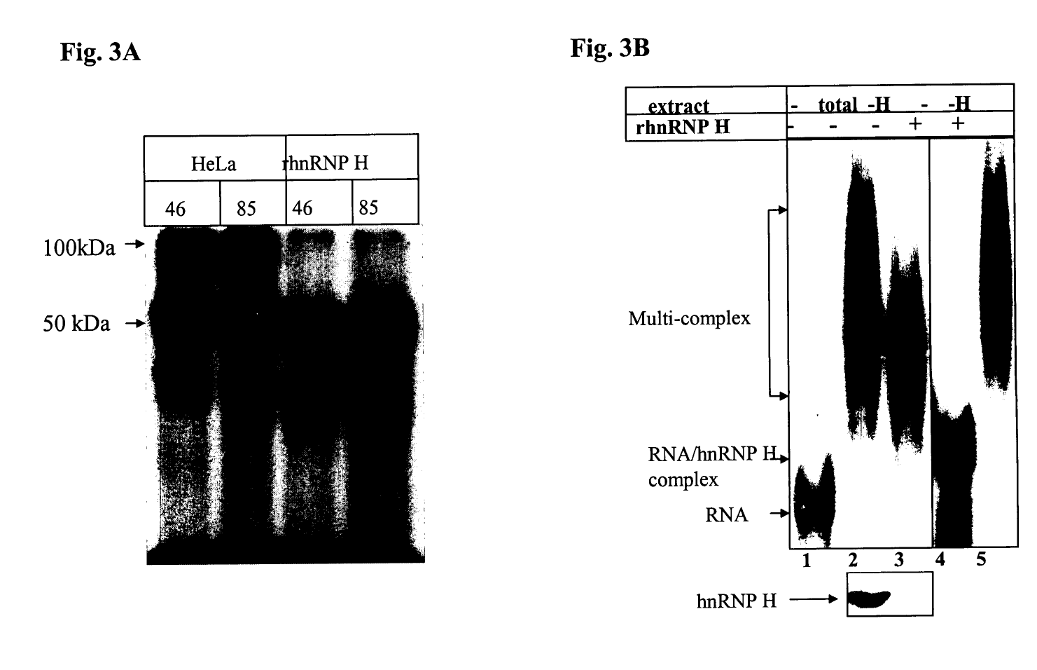

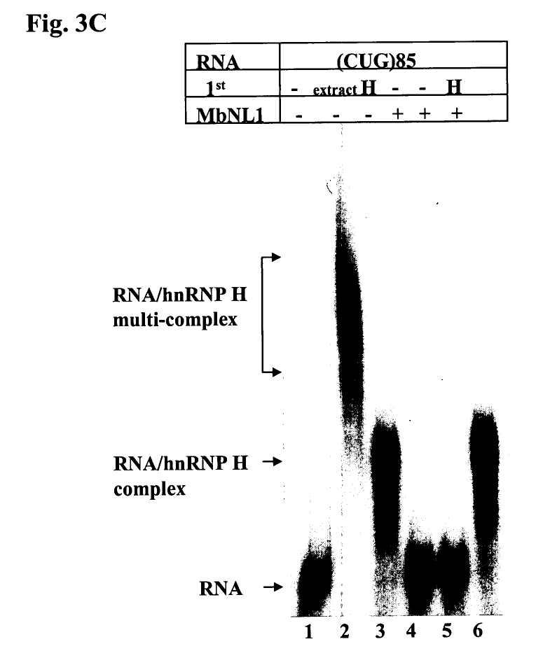

In order to assess binding requirements of hnRNP H with the DMPK-derived RNA containing CUG repeats, we produced a purified recombinant hnRNP H in bacteria. This protein was capable of forming the 50 kDa complex in our crosslinking assays but did not form the 100 kDa complex (FIG. 3A, lanes 3 and 4). These results suggest that the recombinant protein may lack essential post-translational modifications or the dimerization requires one or more nuclear co-factors.

We next immuno-depleted, a HeLa total cell extract depleted of endogenous hnRNP H was prepared using anti-hnRNP H antibody conjugated beads. Complex formations were resolved in a native gel assay (FIG. 3B). A Western blot analysis was performed to assess the depletion of endogenous hnRNP H from the extracts (FIG. 3B, bottom panel). When hnRNP H was depleted from the protein extract, there was a reduction in the high less high molecular weight complex (FIG. 3B, lane 3). When the depleted extract was reconstituted with recombinant hnRNP H, the formation of the larger complex was restored (FIG. 3B, lane 5). These results indicate that the recombinant protein is capable of binding to substrate RNA, but requires additional cellular factor(s) for the formation of large complexes. MBNL1 and 2 are CUG repeat binding proteins that co-localize with the foci that contain mutant DMPK mRNAs with large CUG repeats (Miller et al., 2000; Fardaei et al., 2002). We next sought to determine if recombinant MBNL1 could facilitate dimerization of hnRNP H. But, as it can be seen in lane 4 of FIG. 3C, recombinant MBNL1 had no effect on hnRNP H binding or dimerization.

### Example 5

### Binding of hnRNP H Requires the CUG Repeats and a Splicing Branch Point

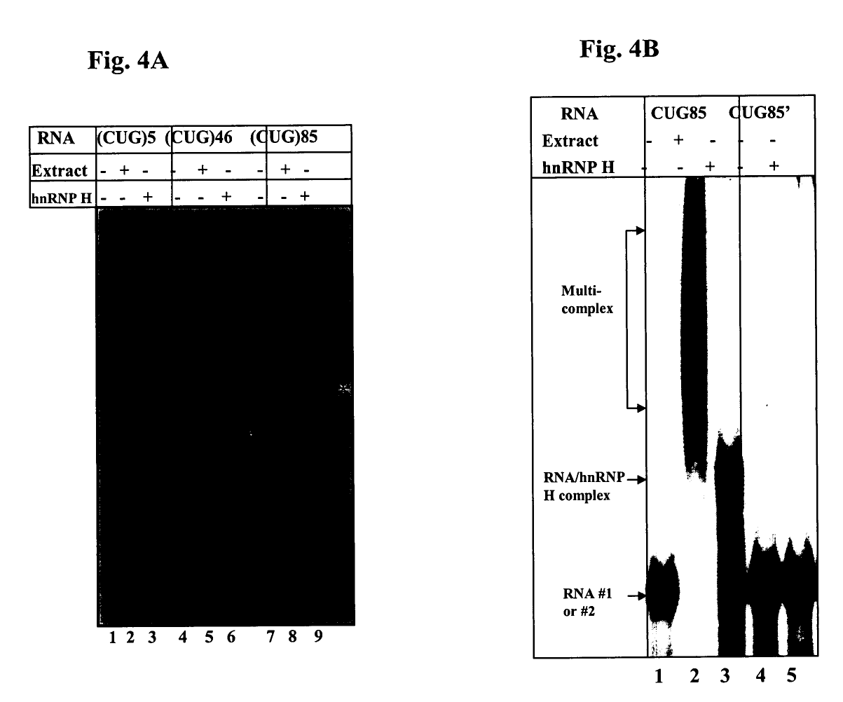

Based on previous data FIGS. 1B, 1C, 2A and 2B) we speculated that the binding of hnRNP H to CUG repeats is proportional to the length of the repeats. The recombinant form of hnRNP H was indubated with different length CUG repeats and complexes resolved in a native gel (FIG. 4A). No binding to the CUG 5 RNA was observed (Lanes 1 and 3). When the CUG 46 RNA was used in this assay, only a small fraction of the protein was gel shifted (marked as * in lane 6). In contrast, the CUG85 RNA was much more gel shifted (marked as * in lane 9). Similar patterns of complex formation were observed in each RNA incubated with total cells extracts (Lanes 5 vs. 8). Combined all hnRNP H binds to CUG repeats in a proportional way depends on the length of the (CUG) repeats.

The different RNAs used in these assays have CUG repeats as well as a splicing acceptor site derived from a downstream of the DMPK gene (Gourdon, 1997). To understand the role of this 3′ branch site in the binding reaction, a mutant CUG85 clone containing a mutant form of the branch site was created (marked as CUG85′, FIG. 1A). Interestingly, the binding of hnRNP H was abolished when the mutant branch site containing probe was used (FIG. 4B, lanes 4 and 5). When the mutated RnA was used for the binding assay with the total cell extract, the formation of the large complex was also reduced. Our results indicate that CUG repeats and the splicing branch point of Exon 16 are both necessary for hnRNP H binding to the transcripts.

### Example 6

### HnRNP H Co-Localizes to CUG Repeat RNAs In Vivo

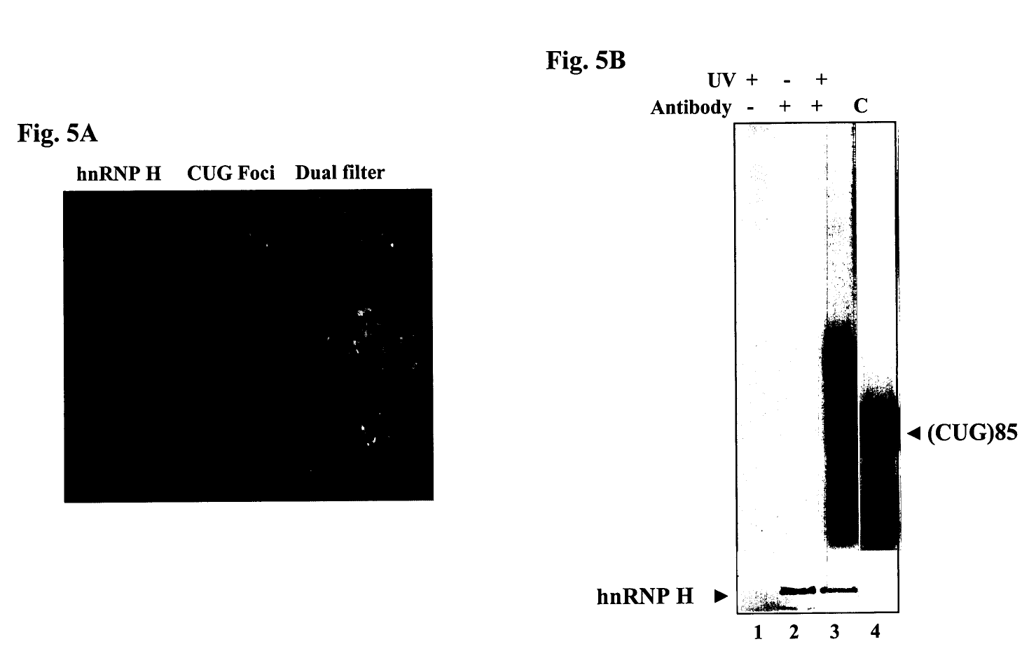

We next wanted to ascertain whether endogenous hnRNP H co-localizes with CUG repeats in patient-derived DM1 cells expressing mutant DMPK transcripts with 750 CUG repeats. An in situ hybridization was performed to reveal both the mutant transcripts (red) and endogenous hnRNP H (green) (see Materials and Methods) (FIG. 5A). Although there was some apparent hnRNP H co-localization with the foci, much of the hnRNP H immunostaining was randomly dispersed throughout the cell nucleus. To determine if binding of hnRNP H to the mutant DMPK transcripts in vivo, a UV crosslinking assay was carried out on DM1 cells (FIG. 5B). DM1 myoblasts were UV-irradiated to cross-link protein-RNA interactions and hnRNP H was immuno-purified using the anti-hnRNP H antibody. No hnRNP H was immuno-purified using the pre-immune sera (FIG. 5B, lane 1, bottom panel), whereas hnRNP H anti-sera precipitated hnRNP H from both irradiated and non-irradiated cells (FIG. 5B, lanes 2 and 3, bottom panel). A phenol extraction was then performed on equal volumes of each immuno-purified sample to isolate bound RNA. The purified RNA was separated in a denaturing gel and hybridized with a radioactively-labeled (CAG)10 probe (FIG. 5B, top panel). Mutant DMPK mRNA was detected in UV-irradiated samples that were immuno-purified with hnRNP H anti-serum. When the parallel experiment was carried out using normal myoblast cells, no bound RNA was detected in UV-treated immuno-precipitated samples. These results are consistent with recent findings by Thornton and colleagues who demonstrate that hnRNP H and F co-localize, to a limited extent, with nuclear foci-containing poly-CUG RNA in DM1 patient brain neurons (Jiang et al., 2004).

### Example 7

### RNAi-Mediated Knockdown of hnRNP H Expression Rescues CUG Repeat-Containing RNAs from Nuclear Retention

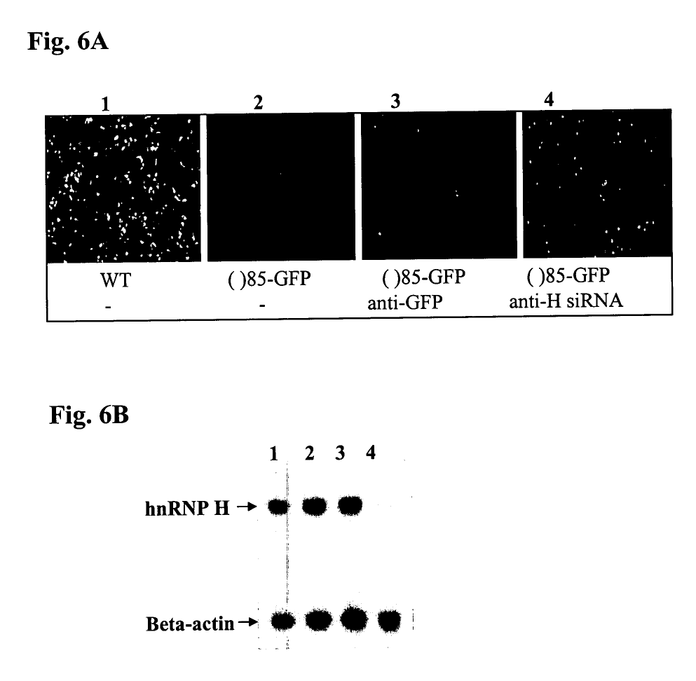

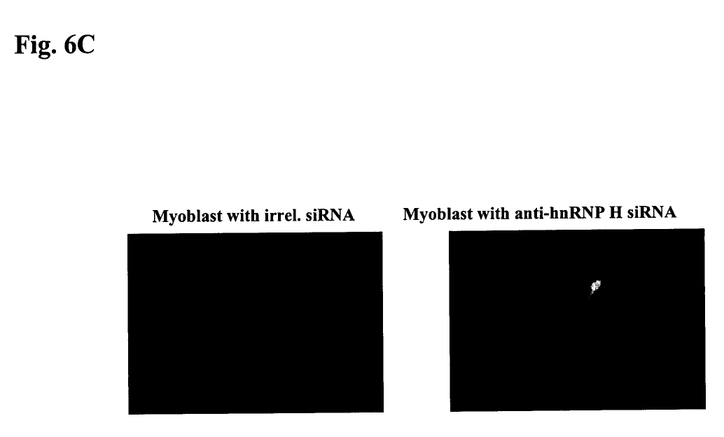

It has been previously shown that expression of an EGFP gene fused to expanded CTG repeats in myoblasts results in a displays severe reduction of EGFP expression due to nuclear retention of the transcripts (Amack and Mahadevan, 2001). If hnRNP H is in part responsible for nuclear retention, one would expect that knocking-down expression of hnRNP H should restore the EGFP-CTG repeat reporter gene expression. To test this possibility, 293T cells were treated with an anti-hnRNP H siRNA (FIG. 2D). The anti-hnRNP H siRNA or an irrelevant (scrambled) siRNA were co-transfected into 293 cells with the eGFP-(CTG)5 (FIG. 6A, panel 1) or eGFP-(CTG) 85 constructs (FIG. 6A, panels 2-4). Transfection of the EGFP-(CTG)5 reporter plasmid in 293T cells results in strong EGFP expression indicating that transcripts with only 5 CUG repeats are readily exported to the cytoplasm and translated (FIG. 6A, panel 1). In contrast the reporter plasmid encoding EGFP-(CTG)85 transfected in the presence or absence of an irrelevant siRNA resulted in only low labels of EGFP expression (FIG. 6A, panel 2 and 3). However, EGFP expression was restored when siRNAs directed against hnRNP H were co-transfected with the EGFP-(CTG)85 (FIG. 6A, panel 4).

In presence of the irrelevant siRNAs, eGFP-(CTG)5 expression was robust whereas eGFP-(CTG)85 expression was severely inhibited (FIG. 6A, panels 1 and 2). However, EGFP expression was restored when anti-hnRNP H siRNAs were co-transfected into these cells (FIG. 6A, #3). The result of this experiment suggests that hnRNP H is a prominent factor in the nuclear retention process of mutant DMPK mRNAs. To insure that the rescue of EGFP expression was a direct effect of the suppression of hnRNP H, aliquots of each cell sample were processed to prepare total RNAs. Northern analysis confirmed downregulation of hnRNP H mRNA by the specific siRNA(FIG. 6B). Parallel experiments were performed using normal myoblasts, but transfection efficiency was less than 20%. Myoblast were co-transfected with the EGFP-(CTG)85 reporter gene and each of the siRNAs (FIG. 6C). When the myoblasts were transfected with the EGFP-(CTG)85 reporter gene and an irrelevant siRNA, the level of EGFP expression was very low (FIG. 6C, left panel). In contrast, cells transfected with the anti-hnRNP H siRNA resulted in rescue of EGFP expression (FIG. 6C, right panel). These results strongly support a role of hnRNP H in the nuclear retention of DMPK mutant transcripts.

### Example 8

### Drug Screening Using the CUG Repeat-EGFP Fusion Genes

This is a positive cell based assay using the described CUG repeats fused to EGFP clone described in the text of manuscript. When the clone is transfected into cells EGFP is not expressed since the mRNA of fused gene is trapped inside of nucleus since the repeats become the binding target of several proteins including hnRNP H. When the cell is grown in the presence of a small molecule that block the binding of protein the nucleus trapping of RNP complex will be relieved and results in expression of EGFP. Large portion of small molecules are toxic to the cells. This assay eliminates any toxic molecules that block either growth of cells or EGFP expression without further screening. The assay protocol is follows.

1. In day 0, the 293T cells are plated in 30% confluence in the 96 well plates. After the cells are attached to the plate surface, a fixed concentration of small drug library is added to each well either manually using multiple pipetter or through automated robotics.

2. In day 1, the cells in the 96 well plate are transformed with the reporter genes. For each well, 10 ng of CUG85-EGFP fusion plasmid are mixed with 10 ul of Opti media containing 0.2 ul of lipofectamine 2000. The mixture can be pre-formed and applied to each well by multiple pipetter.

3. In day 2, the plate is screened under the fluorescent microscope for the EGFP expressing wells. Each plate takes less than 20 min by visual screening.

4. After the initial screening the candidate molecules are further analyzed for their pharmarcokinetics and biological function. Additional modification is done for further improvement of each candidate's efficacy.

Through this work, we have identified a novel protein involved in binding mutant DMPK mRNA. HnRNP H is not a CUG binding protein per see, since it requires both a CUG expansion containing at least 46 repeats and a distal sequence containing a splicing branch point which products rare splicing isoform harboring (Exon 16) of DMPK that does not contain the CUG repeats (Jiang et al., 2004). Under our experimental conditions, we did not detect the binding of other known CUG binding proteins such as the Muscleblind family members or CUG-BP1. Some of the reasons for this could be the use of HeLa cell extracts in conditions that did not favor optimum expression of these proteins, the use of over 10 times non specific RNA competitor or simply our protein-RNA crosslinking method. However, our data show that binding of hnRNP H to mutant DMPK derived RNAs is specific and greatly enhanced by cellular factors present in cell extracts that have yet to be identified. In the presence of these factors, we observed the formation of a complex migrating around 100 kDa in the native gels assays. This complex is absent from extracts made from cells transfected with small interfering RNAs directed against hnRNP H or when the extracts have been depleted of endogenous hnRNP H. These results suggest the formation of a complex comprised of the mutant RNA, hnRNP H and unidentified docking molecules present in our protein extracts. The formation of such a large complex in the cell nucleus could be linked to the DMPK mRNA-containing foci present in DM1 cells.

For several years mouse models of DM1 have been used to elucidate factors involved in DM1 muscle pathogenesis. The first model to show the true involvement of a CUG expansion in DM1 was developed by the Thornton (Mankodi et al., 2000) and Gourdon (Seznec et al., 2001; Gourdon et al., 1997) laboratories. These mice either expressed the human skeletal actin gene fused to CTG repeats or the complete human DM1 locus. The mice in both these models developed several of the hallmark clinical symptoms of DM1 such as myotonia and myopathy. The typical formation of nuclear foci formation was also observed in histological sections. What these mice lacked however was the characteristic muscle weakness and wasting of DM1 patients suggesting other factors are involved in the pathogenesis, such as haplo-insufficiency of the DMPK protein, over-expression of CUG-BP1 (Timchenko et al., 2001) or reduced expression of the downstream SIX5 gene in humans (Sarkar et al., 2000). Recently a mouse knock-out of the gene that encodes the MBLN1 protein which binds to CUG repeats (also CCUG repeats typical of myotonic dystrophy type 2) has been made (Kanadia et al., 2003). These mice developed abnormalities in RNA splicing and eye and muscle defects typical of DM1. These mouse model combined with the SIX5 and DMPK knockout mice provide the great majority of symptoms observed in DM1 ranging from the myotonia, to cataract formation and muscle weakness and wasting. However, what is missing from these models is the identification of the factor responsible for mutant DMPK mRNA sequestration in the nucleus of DM1 cells. Some reports have provided evidence that reducing mutant transcript accumulation could restore some molecular features such as the proper alternative splicing of the insulin receptor in DM1 cells in vitro (Furling et al., 2003; Langlois et al., 2003).

Our compelling data that links hnRNP H to DM1 pathogenesis are the RNAi-mediated expression knockdown experiments. Experiments with eGFP RNAs containing expanded CUGs' previously designed by Amack et al. showed that these transcripts are retained in the cell nucleus as measured by reduced EGFP expression (Amack and Mahadevan, 2001). Using a similar approach, we showed that EGFP expression could be restored in cells expressing these mutant RNAs if the level of endogenous hnRNP H is reduced. We performed RNAi experiments in differentiated DM1 myoblasts to assess whether foci number and intensity were reduced in cells depleted of hnRNP H but poor transfection levels and the inability to identify transfected cells did not allow us to obtain conclusive results with these cells (data not shown).

The mechanisms that underlie DM1 and DM2 pathogenesis are proving to be extremely complex and challenging to comprehend. The pathological CTG and CCTG expansions responsible for causing these diseases create a true gain-of-function that alters cellular metabolism in unpredictable ways, from splicing defects to altering gene expression. In both these diseases, RNA and protein nuclear sequestration seem to be at the root of most these disturbances. Our data suggest that hnRNP H plays a pivotal role in this process.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

It will be appreciated that the methods and compositions of the instant invention can be incorporated in the form of a variety of embodiments, only a few of which are disclosed herein. Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### BIBLIOGRAPHY

Amack, J. D. and Mahadevan, M. S. (2001). The myotonic dystrophy expanded CUG repeat tract is necessary but not sufficient to disrupt C2C12 myoblast differentiation. Hum Mol Genet, 10, 1879-1887.
Arhin, G. K. et al., (2002). Downstream sequence elements with different affinities for the hnRNP H/H′ protein influence the processing efficiency of mammalian polyadenylation signals. Nucleic Acids Res, 30, 1842-1850.
Bagga, P. S. et al. (1998). DSEF-1 is a member of the hnRNP H family of RNA-binding proteins and stimulates pre-mRNA cleavage and polyadenylation in vitro. Nucleic Acids Res, 26, 5343-5350.
Bhagwati, S. et al. (1996). Identification of two nuclear proteins which bind to RNA CUG repeats: significance for myotonic dystrophy. Biochem Biophys Res Commun, 228, 55-62.
Brook, J. D. et al. (1992). Molecular basis of myotonic dystrophy: expansion of a trinucleotide (CTG) repeat at the 3′ end of a transcript encoding a protein kinase family member. Cell, 69, 385.
Bunin, B. A. and Ellman, J. A. et al. (1992). “A general and expedient method for the solid-phase synthesis of 1,4-benzodiazepine derivatives.” J Am Chem Soc 114:10997-10998.
Buratti, E. et al. (2004). hnRNP H binding at the 5′ splice site correlates with the pathological effect of two intronic mutations in the NF-1 and TSHbeta genes. Nucleic Acids Res, 32, 4224-4236.
Caputi, M. and Zahler, A. M. (2002). SR proteins and hnRNP H regulate the splicing of the HIV-1 tev-specific exon 6D. EMBO J, 21, 845-855.
Carell, E. et al. (1994a). “A Novel Procedure for the Synthesis of Libraries Containing Small Organic Molecules.” Angew Chem Int Ed Engl 33:2059-2061.
Carell, E. et al. (1994b). “A Solution-Phase Screening Procedure for the Isolation of Active Compounds from a Library of Molecules.” Angew Chem Int Ed Engl 33:2061-2064.
Caskey, C. T. et al. (1996). Myotonic dystrophy: discussion of molecular mechanism. Cold Spring Harb Symp Quant Biol, 61, 607-614.
Charlet, B. N. et al. (2002). Loss of the muscle-specific chloride channel in type 1 myotonic dystrophy due to misregulated alternative splicing. Mol Cell, 10, 45-53.
Chen, C. D. et al. (1999). Binding of hnRNP H to an exonic splicing silencer is involved in the regulation of alternative splicing of the rat beta-tropomyosin gene. Genes Dev, 13, 593-606.
Cho, C. Y. et al. (1993). “An Unnatural biopolymer.” Science. 261:1303-1305.
Cull, M. G. et al. (1992). “Screening for Receptor Ligands Using Large Libraries of Peptides Linked to the C Terminus of the lac Repressor.” Proc Natl Acad Sci USA 89:1865-1869.
Cwirla, S. E. et al. (1990). “Peptides on Phage: A Vast Library of Peptides for Identifying Ligands.” Proc Natl Acad Sci USA 87:6378-6382.
Dansithong, W. et al. (2005). MBNL1 is the primary determinant of focus formation and aberrant IR splicing in DM1. J Biol Chem, 280, 5773-5780.
Devlin, J. L. et al. (1990). “Random Peptide Libraries: A Source of Specific Protein Binding Molecules.” Science 249:404-406.
DeWitt, S. H. et al. (1993). “Diversomers”: An Approach to Nonpeptide, Nonoligomeric Chemical Diversity.” Proc Natl Acad Sci USA 90:6909-6913.
Erb, E. et al. (1994). “Recursive Deconvolution of Combinatorial Chemical Libraries.” Proc Natl Acad Sci USA 91:11422-11426.
Fardaei, M. et al. (2001). In vivo co-localisation of MBNL protein with DMPK expanded-repeat transcripts. Nucleic Acids Res, 29, 2766-2771.
Fardaei, M. et al. (2002). Three proteins, MBNL, MBLL and MBXL, co-localize in vivo with nuclear foci of expanded-repeat transcripts in DM1 and DM2 cells. Hum Mol Genet., 11, 805-814.
Faustino, N. A. and Cooper, T. A. (2003). Pre-mRNA splicing and human disease. Genes Dev, 17, 419-437.
Felici, F. et al. (1991). “Selection of antibody ligands from a large library of oligopeptides expressed on a multivalent exposition vector.” J Mol Biol 222:301-310.
Filippova, G. N. et al. (2001). CTCF-binding sites flank CTG/CAG repeats and form a methylation-sensitive insulator at the DM1 locus. Nat Genet, 28, 335-343.
Fodor, S. et al. (1993). “Multiplexed biochemical assays with biological chips.” Nature 364:555-556.
Fu, Y. H. et al. (1992). An unstable triplet repeat in a gene related to myotonic muscular dystrophy. Science, 255, 1256-1258.
Furling, D. et al. (1999). Insulin-like growth factor I circumvents defective insulin action in human myotonic dystrophy skeletal muscle cells. Endocrinology, 140, 4244-4250.
Furling, D. et al. (2003). Viral vector producing antisense RNA restores myotonic dystrophy myoblast functions. Gene Ther, 10, 795-802.
Gallop, M. A. et al. (1994). “Applications of combinatorial technologies to drug discovery. 1. Background and peptide combinatorial libraries.” J Med Chem 37:1233-1251.
Goodman and Gilman's The Pharmacological Basis of Therapeutics, 11the Ed., McGraw-Hill Cos., New York, 2006.
Gourdon, G. et al. (1997). Moderate intergenerational and somatic instability of a 55-CTG repeat in transgenic mice. Nat Genet, 15, 190-192.
Harper, P. S. (2001). Myotonic Dystrophy. Third Ed., W. B. Saunders, London.
Ho, T. H. et al. (2004). Muscleblind proteins regulate alternative splicing. EMBO J, 23, 3103-3112.
Horwell, D. et al (1996). “‘Targeted’ molecular diversity: design and development of non-peptide antagonists for cholecystokinin and tachykinin receptors.” Immunopharmacology 33:68-72.
Houghten, R. A. et al. (1992). “The use of synthetic peptide combinatorial libraries for the identification of bioactive peptides.” Biotechniques 13:412-421.
Jiang, H. et al. (2004). Myotonic dystrophy type 1 is associated with nuclear foci of mutant RNA, sequestration of muscleblind proteins and deregulated alternative splicing in neurons. Hum Mol Genet, 13, 3079-3088.
Kanadia, R. N. et al. (2003). A muscleblind knockout model for myotonic dystrophy. Science, 302, 1978-1980.
Kim, D. H. et al. (1999). A mutation in a methionine tRNA gene suppresses the prp2-1 Ts mutation and causes a pre-mRNA splicing defect in Saccharomyces cerevisiae. Genetics, 153, 1105-1115.
Kino, Y. et al. (2004). Muscleblind protein, MBNL1/EXP, binds specifically to CHHG repeats. Hum Mol Genet, 13, 495-507.
Lam, K. S. (1997). “Application of combinatorial library methods in cancer research and drug discovery.” Anticancer Drug Des 12:145-167.
Lam, K. S. et al. (1991). “A new type of synthetic peptide library for identifying ligand-binding activity.” Nature 354:82-84.
Langlois, M. A. et al. (2003). Hammerhead ribozyme-mediated destruction of nuclear foci in myotonic dystrophy myoblasts. Mol Ther, 7, 670-680.
Lu, X. et al. (1999). Cardiac elav-type RNA-binding protein (ETR-3) binds to RNA CUG repeats expanded in myotonic dystrophy. Hum Mol Genet, 8, 53-60.
Mahadevan, M. et al. (1992). Myotonic dystrophy mutation: an unstable CTG repeat in the 3′ untranslated region of the gene. Science, 255, 1253-1255.
Mankodi, A. et al. (2000). Myotonic dystrophy in transgenic mice expressing an expanded CUG repeat. Science, 289, 1769-1773.
Mankodi, A. et al. (2002). Expanded CUG repeats trigger aberrant splicing of ClC-1 chloride channel pre-mRNA and hyperexcitability of skeletal muscle in myotonic dystrophy. Mol Cell, 10, 35-44.
Markovtsov, V. et al. (2000). Cooperative assembly of an hnRNP complex induced by a tissue-specific homolog of polypyrimidine tract binding protein. Mol Cell Biol, 20, 7463-7479.
Miller, J. W. et al. (2000). Recruitment of human muscleblind proteins to (CUG)(n) expansions associated with myotonic dystrophy. EMBO J, 19, 4439-4448.
Ordway, J. M. and Detloff, P. J. (1996). In vitro synthesis and cloning of long CAG repeats. Biotechniques, 21, 609-610, 612.
Philips, A. V. et al. (1998). Disruption of splicing regulated by a CUG-binding protein in myotonic dystrophy. Science, 280, 737-741.
Remington: The Science and Practice of Pharmacy, 21st Ed., Lippincott Williams & Wilkins, Philadelphia, 2005.
Roberts, R. et al. (1997). Altered phosphorylation and intracellular distribution of a (CUG)n triplet repeat RNA-binding protein in patients with myotonic dystrophy and in myotonin protein kinase knockout mice. Proc Natl Acad Sci USA, 94, 13221-13226.
Sarkar, P. S. et al. (2000). Heterozygous loss of Six5 in mice is sufficient to cause ocular cataracts. Nat Genet, 25, 110-114.
Savkur, R. S. et al. (2001). Aberrant regulation of insulin receptor alternative splicing is associated with insulin resistance in myotonic dystrophy. Nat Genet, 29, 40-47.
Scott, J. K. and J. P. Smith (1990). “Searching for Peptide Ligands with an Epitope Library.” Science 249:386-390.
Seznec, H. et al. (2001). Mice transgenic for the human myotonic dystrophy region with expanded CTG repeats display muscular and brain abnormalities. Hum Mol Genet, 10, 2717-2726.
Tapscott, S. J. (2000). Deconstructing myotonic dystrophy. Science, 289, 1701-1702.
Tian, B. et al. (2000). Expanded CUG repeat RNAs form hairpins that activate the double-stranded RNA-dependent protein kinase PKR. RNA, 6, 79-87.
Timchenko, L. T. and Caskey, C. T. (1996) Trinucleotide repeat disorders in humans: discussions of mechanisms and medical issues. FASEB J, 10, 1589-1597.
Timchenko, L. T. et al. (1996). Identification of a (CUG)n triplet repeat RNA-binding protein and its expression in myotonic dystrophy. Nucleic Acids Res, 24, 4407-4414.
Timchenko, L. T et al. (2001b). RNA CUG repeats sequester CUGBP1 and alter protein levels and activity of CUGBP1. J Biol Chem, 276, 7820-7826.
Timchenko, N. A. et al. (1999). CUG repeat binding protein (CUGBP1) interacts with the 5′ region of C/EBPbeta mRNA and regulates translation of C/EBPbeta isoforms. Nucleic Acids Res, 27, 4517-4525.
Timchenko, N. A. et al. (2001a). Molecular basis for impaired muscle differentiation in myotonic dystrophy. Mol Cel. Biol, 21, 6927-6938.
Timchenko, N. A. et al. (2004). Overexpression of CUG triplet repeat-binding protein, CUGBP1, in mice inhibits myogenesis. J Biol Chem, 279, 13129-13139.
Tiscornia, G. and Mahadevan, M. S. (2000). Myotonic dystrophy: the role of the CUG triplet repeats in splicing of a novel DMPK exon and altered cytoplasmic DMPK mRNA isoform ratios. Mol Cell, 5, 959-967.
Zhang, G. et al. (1996). “An enhanced green fluorescent protein allows sensitive detection of gene transfer in mammalian cells.” Biochem Biophys Res Commun 227:707-711.
Zuckermann, R. N. (1994). “Discovery of nanomolar ligands for 7-transmembrane G-protein-coupled receptors from a diverse N-(substituted)glycine peptoid library.” J Med Chem 37:2678-2685.

What is claimed is:

1. A method of screening for modulators of hnRNP H expression comprising: (a) providing a cell or cells in which an hnRNP H promoter directs the expression of a polypeptide; (b) contacting said cell or cells with a candidate modulator; and (c) measuring the effect of said candidate modulator on said polypeptide, wherein a difference in expression of said polypeptide, as compared to untreated cell or cells, indicates that said candidate modulator is a modulator of hnRNP H expression.

2. The method of claim 1, wherein said modulator decreases expression of the polypeptide.

3. The method of claim 1, wherein said polypeptide is reporter or marker polypeptide.

4. The method of claim 1, wherein said cell is a myocyte.

5. The method of claim 1, wherein said cells are differentiated myoblasts.

6. A method of screening for modulators of hnRNP H mutant DMPK binding activity comprising: (a) providing an active hnRNP H preparation; (b) contacting said hnRNP H preparation with a candidate modulator; and (c) measuring the mutant DMPK binding activity of said hnRNP H preparation, wherein a difference in mutant DMPK binding activity of said hnRNP H preparation, as compared to an untreated hnRNP H preparation, indicates that said candidate modulator is a modulator of hnRNP H mutant DMPK binding activity.

7. The method of claim 6, wherein said method is performed in a cell free assay.

8. The method of claim 6, wherein said method is performed in a cell or cells.

9. The method of claim 8, wherein said cell is a myocyte.

10. The method of claim 8, wherein said cells are differentiated myoblasts.

11. A method of screening for modulators of hnRNP H nuclear sequestering activity comprising: (a) providing an active hnRNP H preparation; (b) contacting said hnRNP H preparation with a candidate modulator; and (c) measuring the nuclear sequestering activity of said hnRNP H preparation, wherein a difference in nuclear sequestering activity of said hnRNP H preparation, as compared to an untreated hnRNP H preparation, indicates that said candidate modulator is a modulator of hnRNP H nuclear sequestering activity.

12. The method of claim 11, wherein the nuclear sequestering activity is measured using an mRNA having GUC extension repeats

13. The method of claim 12, wherein the mRNA is a mutant DMPK mRNA.

14. The method of claim 12, wherein the mRNA is mRNA of a reporter or marker gene modified to contain GUC extension repeats.

15. A method of producing a modulator of hnRNP H expression comprising: (a) providing a cell or cells in which a hnRNP H promoter directs the expression of a polypeptide; (b) contacting said cell or cells with a candidate modulator; (c) measuring the effect of said candidate modulator on said polypeptide, wherein a difference in expression of said polypeptide, as compared to untreated cell or cells, indicates that said candidate modulator is a modulator of hnRNP H expression; and (d) producing said modulator.

16. A method of producing a modulator of hnRNP H mutant DMPK binding activity comprising: (a) providing an active hnRNP H preparation; (b) contacting said hnRNP H preparation with a candidate modulator; (c) measuring the mutant DMPK binding activity of said hnRNP H preparation, wherein a difference in mutant DMPK binding activity of said STARS preparation, as compared to an untreated hnRNP H preparation, indicates that said candidate modulator is a modulator of hnRNP H mutant DMPK binding activity; and (d) producing said modulator.

17. A method of producing a modulator of hnRNP H nuclear sequestering activity comprising: (a) providing an active hnRNP H preparation; (b) contacting said hnRNP H preparation with a candidate modulator; (c) measuring the nuclear sequestering activity of said hnRNP H preparation, wherein a difference in nuclear sequestering activity of said hnRNP H preparation, as compared to an untreated hnRNP H preparation, indicates that said candidate modulator is a modulator of hnRNP H nuclear sequestering activity; and (d) producing said modulator.

18. A modulator of hnRNP H expression identified according to the method of claim 1.

19. A modulator of hnRNP H mutant DMPK binding activity identified according to the method of claim 6.

20. A modulator of hnRNP H nuclear sequestering activity identified according to the method of claim 1.

21. A method of treating a subject having a DM1 comprising administering an agent which the modulator of claim 18.

21. A method of treating a subject having a DM1 comprising administering an agent which the modulator of claim 19.

22. A method of treating a subject having a DM1 comprising administering an agent which the modulator of claim 20.


SEQUENCE LISTING (22 sequences)

SEQ ID NO: 1 - DNA, 31 bp, Homo sapiens
ctgctgctgctgctggggggatcacagacca

SEQ ID NO: 2 - DNA, 154 bp, Homo sapiens
ctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctggggggatcacagacca

SEQ ID NO: 3 - DNA, 271 bp, Homo sapiens
ctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctggggggatcacagacca

SEQ ID NO: 4 - DNA, 271 bp, Homo sapiens
ctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctggggggatcagtgacca

SEQ ID NO: 5 - DNA, 22 bp, Artificial
  PCR primer
gaacggggctcgaagcttcctt

SEQ ID NO: 6 - DNA, 32 bp, Artificial
  PCR primer
ctagactggaattcggcttatggtcactgatc

SEQ ID NO: 7 - DNA, 21 bp, Artificial
  PCR primer
cagccatatgctcgagtgatg

SEQ ID NO: 8 - DNA, 20 bp, Artificial
  PCR primer
ctttgttagcagccggatcc

SEQ ID NO: 9 - DNA, 29 bp, Artificial
  Oligonucleotide for synthesis of siRNA
aaggtggagagggattcgtggcctgtctc

SEQ ID NO: 10 - DNA, 29 bp, Artificial
  Oligonucleotide for synthesis of siRNA
aaccacgaatccctctccacccctgtctc

SEQ ID NO: 11 - RNA, 26 nt, Artificial
  Dicer substrate dsRNA sense strand
guuaggaacauuuugaguuacuugaa

SEQ ID NO: 12 - RNA, 27 nt, Artificial
  Dicer substrate dsRNA antisense strand
uucaaguaacucaaauguuccuaacaa

SEQ ID NO: 13 - DNA, 24 bp, Homo sapiens
aagtaactcaaatgttcctaacaa

SEQ ID NO: 14 - protein, 17 aa, Homo sapiens
STGEAFVQFASQEIAEK

SEQ ID NO: 15 - protein, 16 aa, Homo sapiens
HTGPNSPDTANDGFVR

SEQ ID NO: 16 - protein, 19 aa, Homo sapiens
YGDGGSTFQSTTGHCVHMR

SEQ ID NO: 17 - protein, 9 aa, Homo sapiens
VHIEIGPGR

SEQ ID NO: 18 - protein, 13 aa, Homo sapiens
DLNYCFSGMSDHR

SEQ ID NO: 19 - protein, 10 aa, Homo sapiens
VHIEIGPDGR

SEQ ID NO: 20 - protein, 6 aa, Homo sapiens
YVEVFK

SEQ ID NO: 21 - protein, 13 aa, Homo sapiens
DLNYCFSGMSDHR

SEQ ID NO: 22 - protein, 10 aa, Homo sapiens
VHIEIGPDGR